(19) United States
(12) Patent Application Publication
(10) Pub. No.: US 2018/0290957 A1
(43) Pub. Date: Oct. 11, 2018

(54) PROCESS FOR HYDROFORMYLATION OF PENTENOIC ESTERS

(71) Applicant: EVONIK DEGUSSA GMBH, Essen (DE)

(72) Inventors: Stephan BEHRENS, Rostock (DE); Galina Morales TORRES, Rostock (DE); Armin BÖRNER, Rostock (DE); Robert FRANKE, Marl (DE); Detlef SELENT, Rostock (DE)

(73) Assignee: EVONIK DEGUSSA GMBH, Essen (DE)

(21) Appl. No.: 15/939,385

(22) Filed: Mar. 29, 2018

(30) Foreign Application Priority Data
Apr. 11, 2017 (DE) 102017206200.2

(51) Int. Cl. C07C 45/50; C07C 69/66; B01J 31/24

(52) U.S. Cl. CPC C07C 45/50; C07C 69/66; B01J 2531/822; B01J 31/2452; B01J 2231/321; B01J 31/2409

(57) ABSTRACT

Process comprising the process steps of:

a) initially charging a pentenoic ester,
b) adding a ligand of structure 1 or 2:

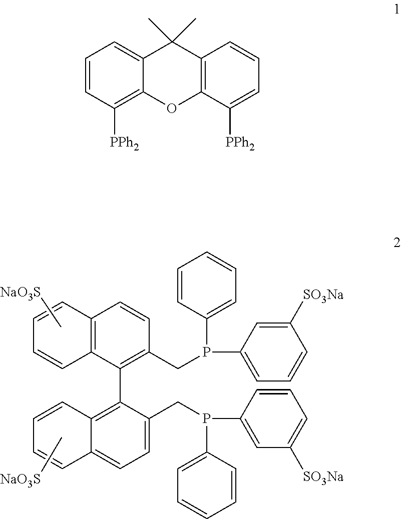

and a compound comprising a metal atom selected from: Rh, Ru, Co, Ir,
c) supplying H₂ and CO,
d) heating the reaction mixture to convert the pentenoic ester to 5-formylpentanoic esters.

The invention relates to a process for hydroformylation of pentenoic esters.

The synthesis of 5-formylpentanoic esters (5-FMP) and of mixtures with the branched aldehydes has often been an object of study for some time now.

U.S. Pat. No. 5,264,616 introduces the use of rhodium complexes with bidentate phosphite ligands. The reaction conditions are 100° C. and 5 bar of synthesis gas. The phosphite ligand with the best performance herein afforded the desired 5-FMP at a conversion of 95.5% with a selectivity of 76.7% after 5 h.

WO95/18089 describes a diphosphite-modified Rh-carbonyl complex. At 90° C. and 10 bar of synthesis gas pressure the best ligand in 27 h afforded a conversion of 54.2% of 5-FMP with a selectivity of 80.4%.

U.S. Pat. No. 6,664,427B1 describes experiments with bidentate phosphoramidites. Employed here, inter alia, was a salicylanilide-based phosphoramidite having a BINOL backbone. The hydroformylation was performed at 100° C. at 10 bar of synthesis gas and afforded 5-FMP with a selectivity of 84.8% and a conversion of 80.3%.

U.S. Pat. No. 6,017,843 likewise describes a hydroformylation reaction. The ligand employed here at 100° C. and 6 bar of synthesis gas affords 5-FMP with 78% n-selectivity at 82% conversion.

WO2014/111446A1 describes a 2-phase catalysis with toluene/H₂O (1:1) as the solvent system. To increase the water solubility of the catalyst, TPPTS ligands were employed. Thus at 100° C. and 10 bar of synthesis gas a selectivity of 92% 5-FMP was achieved. However, the conversion was only 15%.

The technical problem underlying the present invention was that of providing a process in which starting from a pentenoic ester 5-formylpentanoic esters (5-FMP) are produced. Both the yield and the n-regioselectivities herein should be above 85%.

The object is achieved by a process according to claim 1.

Process comprising the process steps of:

a) initially charging a pentenoic ester,

b) adding a ligand of structure 1 or 2:

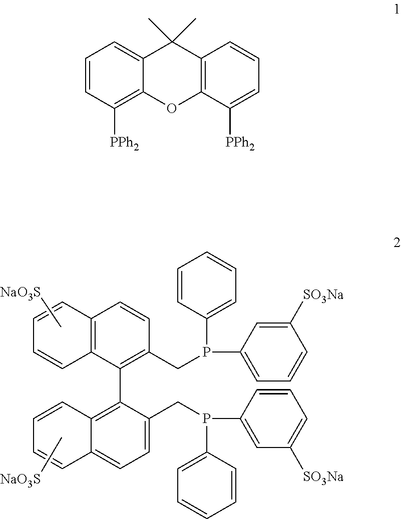

and a compound comprising a metal atom selected from: Rh, Ru, Co, Ir,
c) supplying H₂ and CO,
d) heating the reaction mixture to convert the pentenoic ester to 5-formylpentanoic esters.

In one variant of the process the conversion is effected at a temperature of 80° C. to 130° C. and a pressure of 1 to 20 bar.

In a preferred variant of the process the conversion is effected at a temperature of 90° C. to 120° C. and a pressure of 1 to 15 bar.

In one variant of the process the metal in process step b) is Rh.

In one variant of the process the ligand has the structure 1.

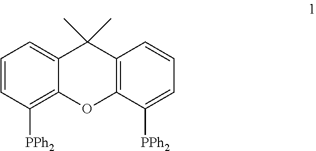

In one variant of the process in which the ligand 1 is employed, the conversion is effected in one phase.

The ligands described in this application form a complex together with a metal atom, for example Rh. This complex then serves as a catalyst for the reactions described in this application.

The conversion “in one phase” is thus a homogeneous catalysis.

In one variant of the process the ligand has the structure 2.

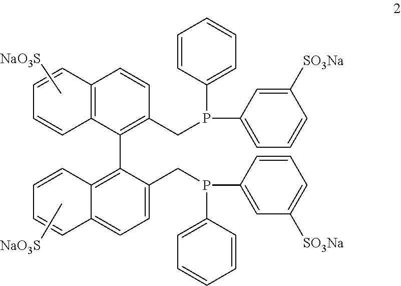

In one variant of the process in which the ligand 2 is employed, the conversion is effected in two phases.

The ligands described in this application form a complex together with a metal atom, for example Rh. This complex then serves as a catalyst for the reactions described in this application.

The conversion “in two phases” is thus a two-phase catalysis.

The invention shall be more particularly elucidated hereinbelow with reference to working examples.

### GENERAL PROCEDURE SPECIFICATIONS

The solvents used were dried using a Pure Solv drying apparatus from Innovative Technology Inc.

NMR spectra were recorded with Bruker AC 250, ARX 300 and AVANCE 500 instruments at 20° C., wherein the signals of the solvent used (CD₂CI₂,H:5.32 ppm) serve as an internal standard. Signal assignment was performed using ¹H experiments and the ¹H spectra of the pure substances. n-Regioselectivity was determined by means of the signals of the aldehyde function protons. These were in the range of 9-10 ppm, wherein the aldehyde group proton of the n-aldehyde is recognizable as a triplet. The signals of the corresponding protons of the i-aldehydes split into doublets and appear at lower chemical shifts.

Gas chromatograms were recorded by means of Hewlett Packard Agilent GC HP6890 and 7890A instruments, both fitted with FI detectors. A calibration to quantify the amounts of substance contained in the substrates and in the reaction products methylvaleric acid and formylpentanoic esters (5-FMP) was also performed and finally used to calculate conversions and yields.

The hydroformylations were performed in a HEL HP Chem-Scan II 8-vessel autoclave fitted with a pressurestat and a thermostat, gas flow measuring means and a magnetic stirrer, and having a respective vessel volume of 20 mL.

Methyl 4-pentenoate (M4P) was used as the substrate for the experiments.

### Performance of the Experiments for Homopeneous Catalysis (One Phase)

For the homogeneously catalyzed experiments the desired ligand is weighed into a suitable Schlenk tube under inert conditions. The ligand is finally dissolved in absolute toluene and admixed with a previously prepared solution of the precursor Rh(CO)₂acac in toluene. The reactor vessels of the autoclave are subsequently purged with argon and charged with the preprepared solutions and the corresponding substrate is added. The reactor vessels are sealed and purged 5 times with argon (pressurized up to 6 bar each time). This is followed by heating to 50° C. and forcing the argon out of the reactor vessel with synthesis gas. This is achieved by 3-fold pressurization with synthesis gas (up to 10 bar) and subsequent decompression. Finally, the reaction solution is brought to reaction temperature and pressurized with synthesis gas until the reported pressure is achieved. The reaction mixture is now stirred for 24 hours at constant temperature and constant pressure. This is followed by slow cooling to room temperature. Samples are taken for analysis.

### Ligands

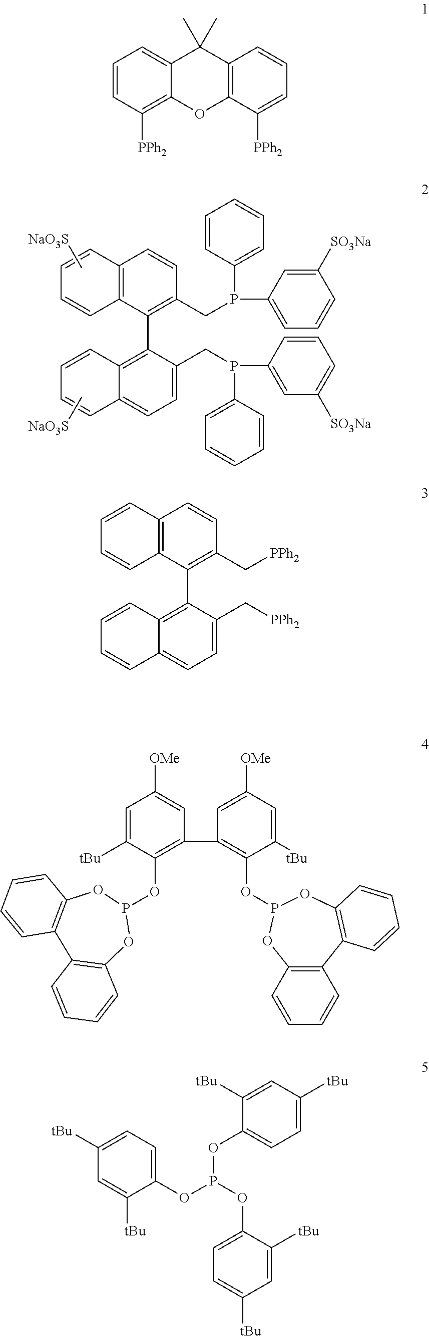

Ligands 1 and 2 are employed in processes according to the invention. Ligands 3, 4 and 5 are comparative ligands.

### Results of the Homogeneous Catalysis (One Phase)

The data and results for the respective homogeneously catalyzed reactions are reported in the tables which follow. The reactions were each performed at 100° C. and 5 bar of pressure. The concentration of the dissolved Rh complex is 100 ppm based on the mole fraction.

### Methyl 4-pentenoate

| Ligand | Rh:L:M4P | Yield [%] | n-regioselectivity [%] |
|---|---|---|---|
| 1* | 1:4:2000 | 90.7 | 91.7 |
| 4 | 1:4:2000 | 68.8 | 87.2 |
| 5 | 1:4:2000 | 78.3 | 46.0 |

*inventive process
L: Ligand
M4P: Methyl 4-pentenoate

### Performing the Two-Phase Catalysis

For the two-phase catalysis initially the desired ligand was weighed into a suitable Schlenk tube under argon. The ligand is subsequently dissolved by addition of deionized water and admixed with the precursor solution. The mixture is thoroughly commixed and subsequently blanketed with absolute toluene. The autoclaves are then prepared as described above and charged. When the reaction mixture is at reaction temperature and pressure it is stirred for 24 hours at constant temperature and constant pressure. After cooling to room temperature samples are taken for analysis.

### Results of the Two-Phase Catalysis

The data and results for the performed reactions are reported in the tables which follow. The reactions were performed at 110° C. and under 10 bar of pressure. The concentration of the dissolved Rh complex is 100 ppm based on the mole fraction.

### Methyl 4-pentenoate

| Ligand | Rh:L:M4P | Yield [%] | n-regioselectivity [%] |
|---|---|---|---|
| 2* | 1:4:2000 | 90.5 | 95.5 |
| 3 | 1:4:2000 | 83.6 | 98.2 |

*inventive process
L: Ligand
M4P: Methyl 4-pentenoate

As is shown by the experiments the problem is solved by the inventive process.

1. Process comprising the process steps of:
a) initially charging a pentenoic ester,
b) adding a ligand of structure 1 or 2: 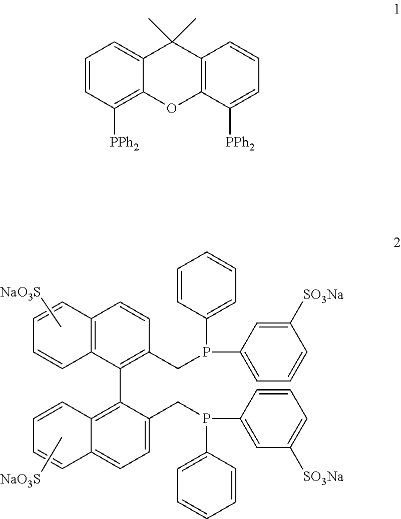
and a compound comprising a metal atom selected from: Rh, Ru, Co, Ir
c) supplying H₂ and CO,
d) heating the reaction mixture to convert the pentenoic ester to 5-formylpentanoic esters.

2. Process according to claim 1, wherein the conversion is effected at a temperature of 80° C. to 130° C. and a pressure of 1 to 20 bar.

3. Process according to claim 1, wherein the conversion is effected at a temperature of 90° C. to 120° C. and a pressure of 1 to 15 bar.

4. Process according to claim 1, wherein the metal in process step b) is Rh.

5. Process according to claim 1, wherein the ligand has the structure 1:

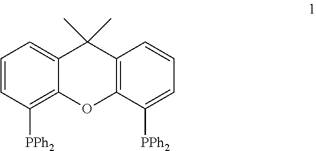

6. Process according to claim 5, wherein the conversion is effected in one phase.

7. Process according to claim 1, wherein the ligand has the structure 2:

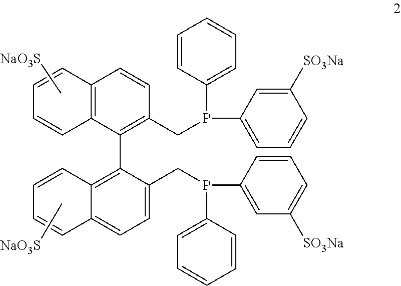

8. Process according to claim 7, wherein the conversion is effected in a two-phase system.
